(19) United States
(12) Patent Application Publication
(10) Pub. No.: US 2014/0236313 A1
(43) Pub. Date: Aug. 21, 2014

(54) MICROSTRUCTURED ADHESION SUBSTRATE FOR ANIMAL OR HUMAN CELLS

(71) Applicants: Martial Balland, Saint Martin D'heres (FR); Kalpana Mandal, Raiganj (IN); Lionel Bureau, Meylan (FR)

(72) Inventors: Martial Balland, Saint Martin D'heres (FR); Kalpana Mandal, Raiganj (IN); Lionel Bureau, Meylan (FR)

(73) Assignees: CENTRE NATIONAL DE LA RECHERCHE SCIENTIFIQUE, PARIS CEDEX 16 (FR); UNIVERSITE JOSEPH FOURIER, GRENOBLE CEDEX 09 (FR)

(21) Appl. No.: 14/234,441

(22) PCT Filed: Jul. 24, 2012
(86) PCT No.: PCT/FR2012/051755

§ 371 Date: Apr. 8, 2014

(30) Foreign Application Priority Data
Jul. 26, 2011 (FR) 11 56814

(51) Int. Cl. A61F 2/02

(52) U.S. Cl. CPC A61F 2/02
USPC 623/23.72

(57) ABSTRACT

The present invention relates to an adhesion substrate for at least one animal or human cell on the surface of which is generated an adhesion pattern for at least one animal or human cell, said pattern including at least two areas adhering to said cell, which are spaced apart by an area not adhering to said cell on which a polymer not adhering to said cell is immobilized, characterized in that the polymer is selected so as to have a conformation which may be modified by varying a parameter of the environment in which the substrate is found, to a method for preparing such a substrate and to the cell analysis methods using such a substrate.

The present invention relates to the technical field of cell biology and of suitable substrates for conducting analysis of cell biology. In particular, the present invention relates to a substrate for adhesion of animal or human cells, and to a method for preparing such a substrate, as well as to an analysis method applying such a substrate, notably with a step for detaching the cell(s) having adhered to the substrate.

At the present time, automated cell analysis techniques for example allowing phenotype screening with a high throughput of cells and the study of the activity of molecules on the activation or inhibition of cell functions are highly sought. One of the goals is to be able to conduct accurate biological analysis while using high throughput automated processes on a large number of cells.

The development of surface microstructuration techniques has led in recent years, to the making of devices with which the adhesion between cells and a solid substrate may be controlled spatially. By controlling the spacing and the geometry of the adhesive areas it is thus possible to immobilize on a surface, regularly distributed individual cells and forced to adopt a shape set by the geometry of the adhesive pattern (EP 1 664 266; Falconnet et at Biomaterials 27 (2006) pp 3044-3063). It is thus possible to prevent the cells from migrating and to orient each cell in the same way.

This type of devices gives the possibility of very specifically studying the effects of physical or biological environmental parameters on the cell response, like the role played by biologically active compounds in the amplification or disabling of specific cell functions. The major benefit of these microstructured adhesive substrates is that they give the possibility of obtaining information on a large population of cells placed in a controlled environment suppressing the shape variability of the cells. It is thus possible to conduct statistical analysis by means of automated tests made possible by the regular aspect of the network of adhesive patterns. These devices are for example used in applications for screening drugs, or further in fundamental studies aiming at determining the impact of an environmental stimulus on intracellular organization, adhesion, division or apoptosis of the cells.

The manufacturing of such microstructure surfaces is based on the elaboration on a substrate of a coating having adhesive patterns separated by regions onto which the cells cannot adhere. Several important criteria have to be taken into account as to the characteristics and properties of these substrates:

the spatial resolution of the adhesive patterns should be of the order of a few micrometers, for patterns with dimensions adapted to the size of individual cell.

there should exist a strong adhesive contrast between the patterns and the remainder of the substrates, in order to effectively control the spatial localization of the cells.

the anti-adhesive areas should ensure their function over durations compatible with cell culture experiments: the coatings should therefore be stable, during their use, over times ranging from a few hours to several days, in the presence of the liquid medium required for cultivating the cells and at a temperature of 37° C.

the surfaces should be stable and remain functional as long as possible, before use, under simple storage conditions, ideally in air and under room temperature conditions.

The elaboration of microstructure substrates for cell biology at the present time is based on two main techniques which are micro-contact printing and photolithography, combined with various surface chemical functionalization methods.

The methods using micro-contact printing as described in patent EP 1 664 266 set into play the following strategy and steps:
1—making an imprint by molding a photosensitive resin for which the relief is the negative of the desired final pattern.
2—making a stamp in elastomer molded on the previous imprint.
3—inking up the stamp by contact with a solution which contains the first species of molecules which are desirably deposited on the surface of the substrate in the form of the final desired pattern.
4—putting the stamp in direct contact with the substrate for transferring molecules.
513 adsorbing on the substrate, in the areas not covered in step 4, a second molecular species. This step is accomplished for example by immersion of the substrates as modified in step 4 in a solution containing the second molecular species.

An exemplary embodiment according to this procedure consists of depositing in step 4, a protein of the extracellular matrix involved in the adhesion, such as fibronectin. The presence of fibronectin according to a regularly organized pattern on the substrate facilitates cell adhesion on this pattern. In order to prevent spreading of the cells outside the pattern, the areas not covered with fibronectin are covered in step 5 with a co-polymer of the PLL-PEG [poly(L-lysine)-poly(ethylene glycol)] type, the PEG block ensuring the anti-adhesive function while the cationic block PLL ensures an electrostatic bond of the molecules with the substrate if the latter is charged negatively.

Alternatives of this procedure consist of depositing, in step 4, the molecular species achieving the anti-adhesive function, which may for example be an alkylsilane or an alkanethiol. A stamp must then be used for which the relief is the negative of the desired final adhesive pattern. Step 5 then consists of adsorbing in the areas which have remained free, an adhesion protein such as fibronectin or collagen.

The micro-contact printing technique has a certain number of advantages such as its versatile nature, the fact that it allows the generation of patterns having micrometric dimensions, the fact that several stamps may be generated from a same mold which may be used several times.

However, the creation of molds requires the use of a clean room (a room or a series of rooms where the particulate concentration is under control in order to minimize the introduction, generation, retention of particles inside). Further, a certain number of additional limitations should be noted, which are:
the deformation of the stamp: the contacting of the stamp with the substrate is a delicate step. Indeed, too high pressure on the stamp may deform the latter and lead to significant distances in the geometry and dimensions of the printed patterns as compared with the original patterns present on the mold.
contamination of the substrate: during the process for baking the mould, fragments may not be baked and may therefore induce defects which will influence the quality of the stamp.
during the breaking of the stamp, the polymer may retract in size and therefore generate a difference between the size of the desired patterns and the (actual) physical size of the latter.
mobility of the ink (the molecule used for generating the patterns): Diffusion of the molecules at the stamp/substrate interface may occur during the step for contacting the substrate. This side mobility (diffusion) may induce spreading of the molecules outside the desired regions (patterns).
Both molecular species deposited on the substrates are most of the time physisorbed, i.e. in a relatively small interaction with a substrate. This may pose stability problems of the adhesive pattern during cultivation of the cells.

The photolithography technique, as for it, uses light, generally in the wavelength range corresponding to UVs (ultraviolet), in order to produce oxygenated reactive species in the vicinity of the surface of a substrate on which it is present a photosensitive chemical group,which will be modified or degraded under the effect of the radiation. This modification is spatially selected when the illumination of the surface occurs through a photomask, used as a “stencil” having transparent areas and opaque regions towards UV radiation.

Adhesive patterns may be obtained by this method by illuminating, through a photomask bearing the desired pattern, an initially anti-adhesive, uniform coating deposited beforehand on a substrate. The induced chemical modifications will allow adsorption on the illuminated areas, of adhesion proteins. The light may also be used for detaching or destroying the anti-adhesive molecular species, and thus allow subsequent deposition, at the illuminated locations, of proteins or ligands allowing adhesion of cells.

An example for making microstructured substrates according to this method is based on the adsorption, on a glass surface, of a thin PLL-PEG layer. This layer, once the solvent used for the deposition has been cleared, is put into contact, (direct contact or via a contact liquid) with a photomask bearing the desired pattern. The illumination of the surface for a few minutes with deep UV (a wavelength of less than 200 nm) has the effect of generating carboxyl groups on the PEG macromolecules. These carboxyl groups then allow adsorption of fibronectin, which promotes adhesion of the cells to the locations of the coating which have been insolated (Azioune et al. Lab On a Chip 9 (2009) pp 1640-1642).

The photolithography technique is a rapid technique and easy to apply. It does not require the use of a clean room. It allows adhesive patterns to be made with micrometric resolution reliably and reproducibly. The photomasks, which are customizedly available commercially, are reusable a very large number of times. Nevertheless, this technique also has problems related to the stability of microstructured coatings which are most often formed by simple physisorption of a molecular species on the substrate.

Moreover, regardless of the microstructuration technique used, when the analyses carried out on the cells immobilized on the substrate have to be followed with tests practiced in a solution, which is very frequent, it is indispensable to be able to detach from the surface the studied population of cells, in order to suspend the cells. For this purpose, it is necessary, with all the present microstructured substrates, to resort to enzymatic or chemical methods. Thus, conventionally trypsin is used, an enzyme which degrades the proteins involved in cell adhesion, or ethylene diamine tetra-acetic acid (EDTA), which chelates the calcium ions required for having integrins operate, transmembrane proteins involved in the adhesion. Both of these methods may however affect cell viability and represent “aggressive” detachment methods which may affect the results of the tests which follow the detachment of the cells.

Making microstructured substrates and giving the possibility of studying adhered cells on the one hand and in a second phase the detachment of the latter by a less invasive technique than the use of trypsin or EDTA, would represent significant progress.

One of the goals which the present invention proposes to reach, is the one which proposes an adhesion substrate for at least one animal or human cell defining a surface on which at least one adhesion pattern is present for at least one animal or human cell, said pattern including at least two adhesive areas to said cell which are spaced from each other by a non-adhesive area to said cell on which a non-adhesive polymer to said cell is immobilized, characterized in that the polymer is selected so as to have a conformation which may be modified by varying a parameter of the environment in which substrate is found. In particular, the polymer is selected so as to define a layer, for which the thickness maybe increased relatively to a reference thickness measured when the substrate is placed in pure water at 37° C. and in the absence of light, by modification of a parameter of the environment in which the substrate is found.

Within the scope of the invention, advantageously, the adhesive areas of an adhesive pattern are positioned so that the cell may adhere under adhesion conditions onto the adhesive areas by forming a bridge on the non-adhesive polymer and in that the non-adhesive polymer appears in a so called compacted conformation, under the conditions of adhesion of the cell, in order to be deployed in a so-called deployed conformation by modifying a parameter of the environment in which the substrate is found.

The object of the invention is also the whole of the substrate and methods as defined in the claims.

The invention notably relates to a method for detaching in vitro at least one cell adhered beforehand onto the adhesive pattern(s) of a substrate according to the invention in which detachment of the cell is achieved by varying an environmental parameter in which the substrate is found, causing deployment of the non-adhesive polymer.

The description which follows, with reference to the appended Figures, gives the possibility of describing the invention in a detailed way.

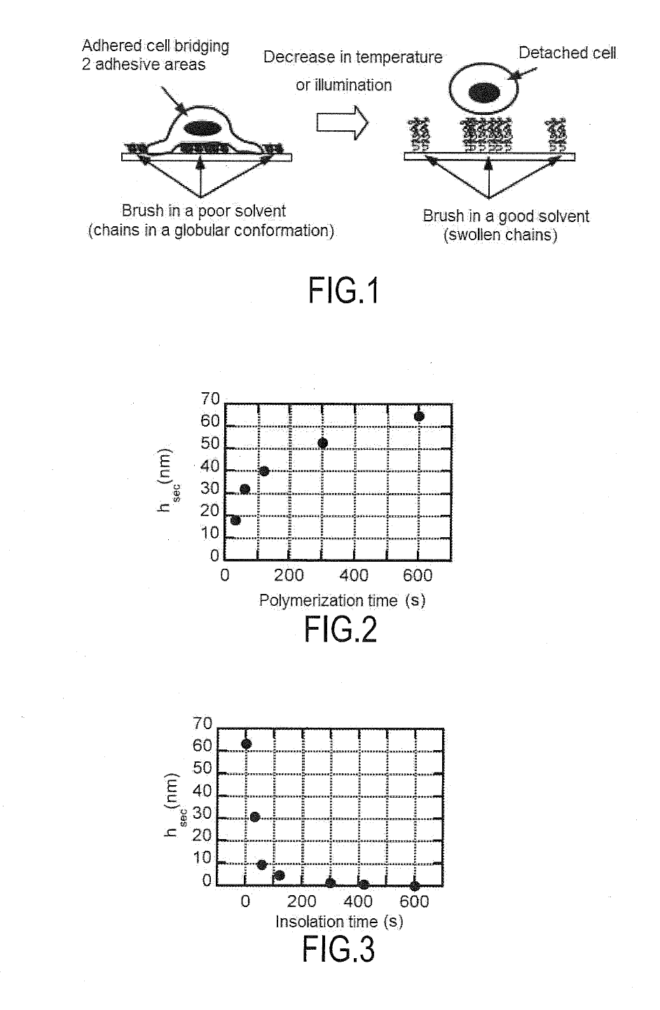

FIG. 1 shows a block diagram allowing illustration of how a device according to the invention allows detachment of a cell which has adhered onto an adhesive pattern, by varying an environmental parameter. On the left, a cell adhered on the substrate and forming a bridge above a non-adhesive area of polymer is in a so-called compacted conformation also called a globular conformation. On the right, after varying the environmental parameter, the polymer micromolecules adopt a deployed conformation, also called a swollen conformation, which causes detachment of the cell.

FIG. 2 shows the dependency of the dry thickness of poly(N-isopropylacrylamide) (PNIPAM) brushes obtained in the examples, with the polymerization time. Initial APTES concentration during the first grafting step: 2.10⁻⁴ M.

FIG. 3 shows the dependency of the dry thickness of poly(N-isopropylacrylamide) (PNIPAM) brushes obtained in the examples versus insolation time with deep UV.

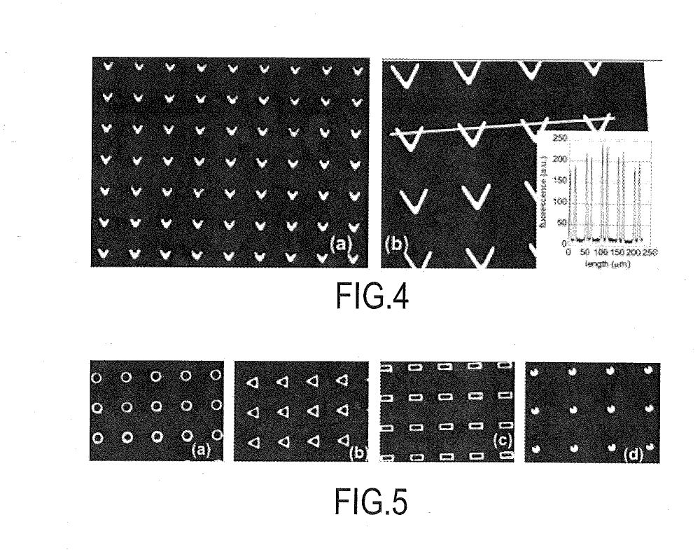

FIG. 4 shows (a) V-shaped adhesive patterns marked in fluorescence by adsorption of fibronectin/fibrinogen-alexa fluor, observed at 546 nm. Size of the image: 895×676 μm². (b) V-shaped adhesive patterns marked in fluorescence by adsorption of fibronectin/fibrinogen-alexa fluor, observed at 546 nm. Insert: fluorescence intensity profile along the line traced on the image. Size of the image: 420×315 μm².

FIG. 5 shows examples of shapes of adhesive patterns, in the form of a ring (a); of a triangle (b); of a rectangle (c); of a “pacman” (d); marked in fluorescence by adsorption of fibronectin/fibrinogen-alexa fluor, observed at 546 nm.

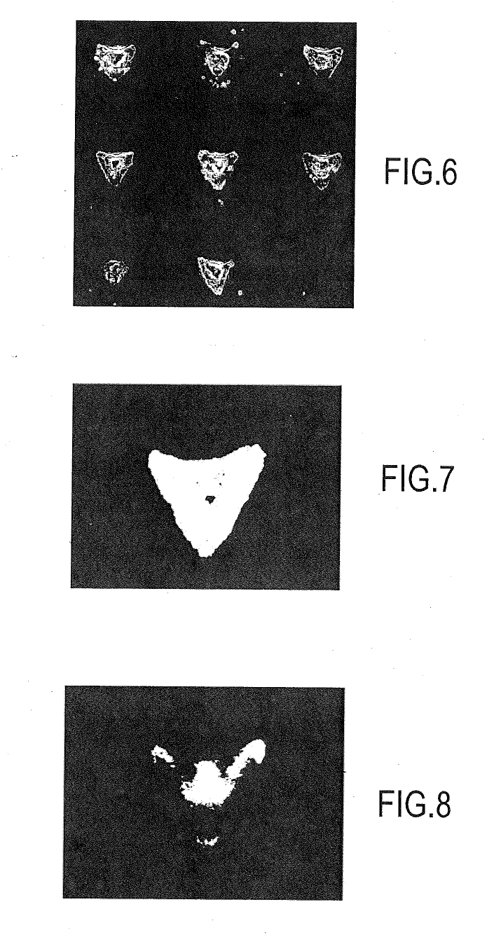

FIG. 6 is a phase contrast image of a network of REF52 cells adhered on V-shaped patterns. Cell cultivation carried out on a substrate on which fibronectin was adsorbed beforehand. Size of the image: 270×270 μm².

FIG. 7 is an image of a single REF52 cell. The fluorescence signal uniformly detected in the cell, corresponds to the paxillin-YFP marking.

FIG. 8 is an average image of a paxillin-YFP marking built from 11 images of individual REF52 cells. The image shows the aggregation of paxillin at the apices of the V-pattern on which the cells have adhered. The central spot corresponds to the position of the cell nucleus: the strong thickness in this region causes saturation of fluorescence signal.

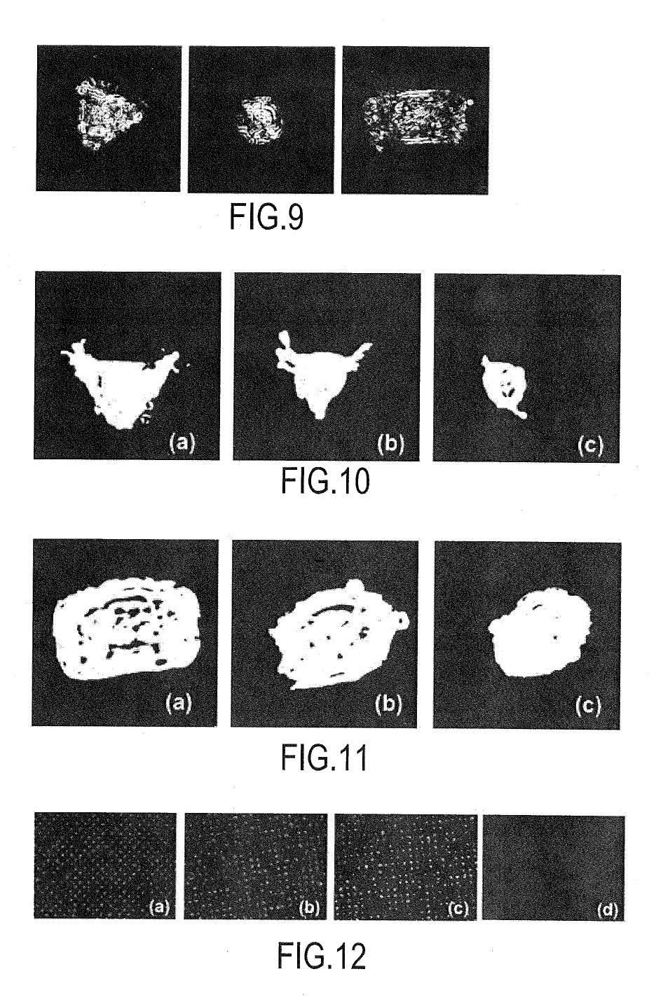

FIG. 9 shows images in bright field microscopy obtained on MEF cell cultures adhered on different patterns and made without any prior ligand adsorption. Left: triangle containing a central area covered with PNIPAM. Center: ring. Right: rectangle containing a central area covered with PNIPAM.

FIG. 10 shows bright field images of an REF52 cell initially adhered on a V-pattern. (a): immediately after setting the temperature to T=30° C. (b) 36 minutes after lowering the temperature. (c) 47 minutes after the latter. Size of the images: 85×85 μm².

FIG. 11 shows bright field images of an MEF cell initially adhered on a rectangular pattern. (a): immediately after setting the temperature to T=30° C. (b) 10 minutes after lowering the temperature. (c) 25 minutes after the latter. Size of the images: 55×55 μm².

FIG. 12 shows phase contrast images of MEF cell networks adhering onto a microstructured substrate. (a): first use of the substrate. (b): second cell structure on the same substrate, after detachment of the cells of the first culture. (c): third culture of cells on the same substrate, after detachment of the cells of the second culture. The bright spots correspond to the adhered individual cells. (d): condition of the surface of the substrate between two cultures. No cell is adhered after setting room temperature and rinsing the substrate. Size of the images: 2,200×1,664 μm².

The substrates according to the invention are designed so as to allow (i) a cell to adhere onto an adhesive pattern, and (ii) this cell of the substrate to be then detached by varying an environmental parameter such as the temperature or the lighting, without resorting to enzymatic or chemical methods such as the use of trypsin or EDTA.

The adhesive pattern has a shape such that a cell will be able to adhere by binding to so-called adhesive areas, but by positioning itself above a non-adhesive area of the pattern. The principle is illustrated in FIG. 1 which shows a cell bound to the adhesive areas of an adhesive pattern. At least one portion of the adhesive areas being separated by a non-adhesive area, the cell once it has adhered on the adhesive areas will form a bridge on the non-adhesive area. The adhesive areas, although at least partly separated by a non-adhesive area, may be connected together in order to form an open shape (case of an L-shape, for example) or a closed shape (case of a ring or polygon for example) or else be isolated from each other (case of a line and one or two dots, for example). The adhesive areas may consist of any bidimensional shape and appear as a line, a strip, a dot, a circular arc . . . . The maximum distance generated by the non-adhesive area separating two adhesive areas of a same pattern, is most often less than 50 μm, preferably in the range from 5 to 20 μm. The adhesive areas of the adhesive patterns may notably form a broken or curved line which may be closed by for example delimiting a polygon or a ring or which may be open with the shape of a V, U, C or L for example or correspond to two or several lines spaced out, or to a line and one or several dots spaced apart. It is notably possible to use adhesive patterns in which the adhesive areas form an anisotropic shape, such as the adhesive patterns notably described in patent EP 1 664 266 to which reference may be made for more details, or such as shown in FIG. 4 or 5.

By “adhesive area”, is meant a cytophilic area, on which an animal or human cell will be able to adhere by adsorption, in particular when the latter is put into contact with the substrate in a suitable aqueous culture medium, at 37° C. and in the absence of light. An adhesive area is an area on which the proteins of the extracellular matrix (fibronectin, laminin, collagen . . . ) secreted by the cells during the cultivation may be adsorbed in order to then allow adhesion of the cells via the integrins (trans-membrane proteins of the cells). In particular, glass, silanized glass, silica or surface-oxidized silicon substrates have adhesive properties for the cells. Moreover, many molecules are known for promoting immobilization of cells. Mention may be made of antigens, antibodies, cell adhesion molecules, extracellular matrix molecules, such as laminin, fibronectin and collagen, synthetic peptides, carbohydrates and the like. The selection of the materials and/or molecules present on these areas will be adapted by one skilled in the art, to the cells for which adhesion is desired.

By “non-adhesive area” is meant a cytophobic area on which the cells do not adhere, in particular when the latter are put into contact with the substrate in a suitable aqueous culture medium, at 37° C. and in the absence of light. The absence of cell adhesion on this area is associated with the fact that the proteins of the extracellular matrix (MEC) mentioned above can neither be adsorbed specifically thereon nor be inserted thereon. The polymer providing the anti-adhesive function is there selected so as to not have any specific interaction with the proteins of the MEC: an electrically neutral polymer will be preferred, and not having any sequences of amino acids which may be recognized by the proteins. The cells which may adhere on a substrate according to the invention may be of any type of animal or human cell. These may notably be fibroblasts, hematopoietic, endothelial or epithelial cells. The cell may be of the wild or modified type. These may be tumoral cells.

Preferably, the adhesive pattern(s) used within the scope of the invention has(have) a size which is adapted for allowing adhesion of a single individual cell. In particular, the adhesive patterns appear as micropatterns which have a size which substantially corresponds to the size of the cell to be immobilized or a size such that only one cell may be spread thereon and may divide, with limited movement. Typically, the surface area of the patterns which are delimited by the adhesive areas in the case when they are all connected or through the envelope connecting the adhesive areas, when the latter are not connected, is from 1 to 2,500 μm², preferably from 1 to 2,500 μm², notably from 1 to 500 μm² or from 500 to 900 μm². For example, the width of the diverse lines, bridges or forms forming the adhesive areas will be from 1 to 20 μm, notably of the order of 10 μm.

Within the scope of the invention the ratio between the non-adhesive area over the total surface area of the pattern is preferably in the range from 30 to 90% and preferentially between 50 and 90%.

Most often, a network of identical adhesive patterns as defined within the scope of the invention will be generated at the surface of the substrate. The substrate therefore has a microstructured surface. Preferably, the adhesive patterns are regularly distributed and spaced apart in the network, as notably illustrated in FIGS. 4 and 5. Such a network will allow adhesion of individual cells to well-determined locations at the adhesive patterns, thus preventing migration of the cells. Advantageously, the patterns will correspond to patterns as described in patent EP 1 664 266 which leads to polarization of the cells which will be placed in a reproducible way on the adhesive patterns. When the substrate includes a network of adhesive patterns, the latter are isolated from each other by non-adhesive regions. Preferably, the average distance separating two consecutive adhesive patterns will be at least equal to twice the maximum distance generated by the non-adhesive area and separating two adhesive areas of a same pattern. Most often, the non-adhesive polymer immobilized on the non-adhesive areas of the adhesive patterns is also immobilized on the non-adhesive regions separating two consecutive adhesive patterns. Most often, the substrate is, in this case, totally covered with a selected polymer so as to have a conformation which may be modified by varying a parameter of the environment in which the substrate is found, as defined within the scope of the invention, except for the adhesive areas of the adhesive patterns.

As an example, a network comprising from 5 to 10,000 adhesive patterns may be made at the surface of the substrate. It is notably possible that 10 to 50,000 adhesive patterns, and preferably 5,000 to 15,000 adhesive patterns, are present per cm² of substrate.

Within the scope of the invention, provision is made for immobilizing on the non-adhesive area of the pattern(s) present on the substrate, a polymer onto which a living cell will not adhere but the conformation of which and therefore the size will be able to be modified by modifying an external parameter. In particular, the selected polymer will have a given conformation and size, under the conditions for adhesion and cultivation of the cell, which will be able to be modified in order to attain deployment or swelling of the polymer, as a result of the modification of an external parameter. Generally, adhesion and cell cultivation are achieved in an aqueous medium at 37° C. Also, as a reference, it is possible to take the size (i.e. the length which the polymer has in its conformation) and the conformation of the polymer in pure water at a temperature of 37° C. The polymer immobilized on the non-adhesive areas is able to undergo modification of its conformation causing an increase in its size, when an external parameter, also called an environmental parameter, is varied, such as temperature or illumination. This increase in the size of the polymer will push the cell, at the bridge which it forms above the polymer, and force it to be detached from the adhesive areas.

The non-adhesive polymer may be a homopolymer or a copolymer, the water solubility of which in depends on an external environmental parameter. Under usual environmental conditions for cell cultivation, i.e. at a temperature of 37° C. and in the absence of light, pure water should be a poor solvent for the polymer immobilized on the substrate, which then adopts a so-called globular or compacted conformation. The detachment of the cultivated cells on the substrate is induced by the change in conformation of the polymer, which passes from its globular shape (poor solvent) to a swollen conformation (with solvent) during the variation of an environmental parameter.

In order to ensure innocuousness of the variation of environments with regard to the cultivated cells, the use of either temperature or electromagnetic radiation preferably corresponding to light radiation close to the visible range, will be preferably used as a parameter for controlling the conformation of the polymer.

The environmental parameter which will be modified, may for example, correspond to a variation in temperature and in particular to a decrease in temperature. In particular, the non-adhesive polymer may be a heat-sensitive polymer having in water a lower solubility critical temperature (LCST) which belongs to the range from 25 to 35° C. The LCST may notably be determined by a measurement, versus temperature, of the adsorption of light by a diluted solution of the polymer in water at a concentration which may range from 0.01 to 1% by mass, the LCST corresponding to the increase in the turbidity of the solution, and is defined as a temperature at which the transmission of the solution passes from 100% to 50%, as described in the publication of T. BALTES et at. Journal of Polymer Science Part A, 37 (1999) pp 2977-2989. As an example of such polymers, mention may be made of poly(N-isopropylacrylamide), poly(oligo(ethylene glycol)methacrylate) and poly(N,N-dimethyl-aminoethyl methacrylate). With such polymers, detachment of the cells is induced when the temperature of the culture medium in which is placed the substrate, is lowered from 37° C. to a temperature below the LCST.

It is also possible that the variation of the environmental parameter corresponds to a variation in the light illumination to which the substrate is subject. In particular, it is possible to use a photo-stimulable polymer, for which the conformation in water at 37° C. passes from a conformation which will be called globular or compacted in the absence of light, to a so-called swollen or deployed conformation, when the latter is illuminated with a particular wavelength. For example, the non-adhesive polymer may be poly{N,N-dimethylaminoethylmethacrylate-co-4-methyl-[7-(methacryloyl)-oxyethyloxy]-coumarin} which has such a property when it is illuminated with a wavelength of 365 nm.

Preferably, within the scope of the invention, the non-adhesive polymer is immobilized on the surface of the substrate through a covalent bond. This notably gives the possibility of guaranteeing stability of the polymer-substrate bond, notably during cell cultivation. In particular, the non-adhesive polymer may be immobilized along a brush of polymeric chains, each of the polymeric chains being bound to the surface of the substrate through one of its ends.

The non-adhesive polymer forms a non-adhesive layer or coating for the cells, the thickness of which may be modified by acting on an environmental parameter. Within the scope of the invention, it is preferable that the non-adhesive polymer be immobilized with a density of polymeric chains belonging to the range from 0.1 to 1 chain per nm², which gives the possibility of optimizing this adhesivity. The presence of the polymer as a brush with such sufficiently high density moreover gives the possibility of preventing, because of steric repulsion, the insertion of the proteins into the anti-adhesive coating.

Preferably, the change in conformation of the polymer, in its reference conformation in water at 37° C. in the absence of light, or in its compacted conformation under the adhesion conditions, to its deployed conformation obtained after variation of the environmental parameter, is associated with an average thickness variation of the layer formed by the polymer by at least a factor 2. The average thickness variations of this layer, and in particular of this layer formed with a brush of polymers, between the compacted conformation and the deployed conformation may for example be characterized by an optical measurement of the average thickness, by ellipsometry in a liquid phase. For example, it is possible to use a non-adhesive polymer which appears as polymeric chains comprising from 50 to 10,000 monomer units.

The substrates used appear as a plate, a blade or lamella generally, conventionally used as an analysis substrate. For example, they will consist of glass, silanized glass, silica or oxidized silicon at the surface and will include surface coatings as explained earlier and hereafter for the formation of adhesive patterns. Such substrates even in the absence of coating are adhesive for the cells. But it is possible that molecules selected from laminin, fibronectin and collagen be immobilized on the adhesive areas of the adhesive patterns.

The object of the present invention is also a method for preparing a substrate according to the invention, in which the selected non-adhesive polymer is immobilized notably through a covalent bond, on the total surface of the substrate and photo-ablation of the non-adhesive polymer is then achieved by photolithography, at the areas corresponding to the adhesive areas of the adhesive patterns. Advantageously, the immobilization of the polymer at the surface may be accomplished by controlled radical polymerization of at least one monomer on an immobilized polymerization initiator, preferably, through a covalent bond at the surface of the substrate.

The thereby obtained anti-adhesive coating is formed with a thin layer of polymer. In order to ensure stability of the coatings, a layer covalently bound to the substrate will be preferred, for example, in the form of a dense brush of polymeric chains. The covalent bond is generally ensured by coupling on Si—OH functions present at the surface of the substrates used, of compounds of the alkoxysilane (for example trimethoxy- or triethoxy-silane) or of the halosilane (for example trichlorosilane) type according to techniques well known to one skilled in the art. This brush may for example be elaborated by means of various techniques:

(i) by grafting polymeric chains bearing a terminal chemical function capable of forming a covalent bond with a group covalently grafted beforehand on the substrate. An example of this method, said to be a “grafting-onto” method, consists of grafting onto the substrate a silane exposing at the surface an Si—H bond, and then of putting the surface into contact with a solution or a melt of polymeric chains having a terminal vinyl function, in the presence of platinum acting as a catalyst. The hydrosilylation reaction which occurs between the Si—H of the surface and the unsaturated bonds at the ends of the chains gives the possibility of covalently binding the macromolecules to the substrate (Bureau et Leger, Langmuir 20 (2004) pp 4523-4529).

(ii) by polymerization initiated from the surface (a so-called “grafting-from” method). The first step of this method consists of grafting onto the substrate a molecule being used as an initiator for the polymerization. The nature of this molecule depends on the selected polymerization method. This may be a silane having the terminal function adapted for initiating polymerization (for example a C—X bond, wherein C is a carbon atom and X is a halogen such as chlorine, bromine or iodine, if the polymerization occurs by radical polymerization with atom transfer, called ATRP). This grafting is typically accomplished by immersing the substrate in glass or in silica for a few minutes to a few hours in a diluted solution of silane in an organic solvent (for example toluene, THF, cyclohexane) at a temperature which may vary from room temperature to about 100° C. Alternatively, a silane having an amine terminal group (NH₂) may be grafted in an aqueous solution onto the substrate. The amine group is then functionalized by reaction with a molecule having the group being used as a polymerization initiator, as described in the example detailed further on. The growth of the polymeric chains is then achieved from this initiator by means of a controlled radical polymerization method such as ATRP, by reversible addition/fragmentation by chain transfer (RAFT), or by polymerization mediated by a nitroxide radical or an iniferter. These different techniques are well known to one skilled in the art and are described in detail by Barbey et al. in the publication, Chem. Rev. 109, 5437-5527 (2009) to which reference may be made for more details. The polymerization reaction is conducted by exposing the substrate bearing the initiator to a solution of the selected monomer(s) in the presence of the agents required for polymerization (catalyst, or RAFT agent, for example).

The method described in (ii) will be preferred since it allows elaboration of brushes of polymeric chains with a controlled size and for which the grafting density (number of polymeric chains per unit surface) is high (typically of the order of 0.1 to 1 chain per nm²), thus ensuring good anti-adhesive properties.

The polymerization conditions, and in particular the polymerization time and the concentration of grafted molecules at the surface (polymer in the case (i) and polymerization initiator in the case (ii)), will be adapted by one skilled in the art depending on the desired density and on the thickness of the polymeric layer.

Next, from a polymer coating obtained according to the techniques above, which uniformly covers the surface of the substrate, the polymer coating is exposed, through a photolithography mask bearing the desired patterns, to ultraviolet radiation in a wavelength range, most often below 200 nm (deep UV). This leads to photo-ablation of the polymer at the areas of the mask which are transparent to the UV. An exposure of a few minutes leads to total ablation of the polymer, which gives the possibility of exposing the underlying substrate, onto which cells adhere because of the nature of the material which makes it up.

Although the above method is preferred, adhesive patterns on the substrate may be made by any techniques with which it is possible to obtain a surface on which the adhesive areas are separated by regions covered with the anti-adhesive polymer coating. It is notably possible to use printing by micro-contact in order to deposit on the substrate, the molecule being used as an initiator for the polymerization according to a negative pattern of the desired final adhesive pattern. The growth of the macromolecules is then limited to the areas of the functionalized surface during buffering, moreover leaving the substrate untreated. Nevertheless, this method is not preferred since it has the drawbacks of the micro-contact printing technique which was detailed in connection with the prior art.

Regardless of the technique used for grafting the non-adhesive polymer to the desired locations, it is possible to adjust the specificity of the adhesion of the adhesive areas of the substrate by adsorbing in the areas not covered with the non-adhesive polymer, a ligand involved in cell adhesion, for example a protein such as fibronectin, collagen or laminin. In particular, in the case of the method detail in (i), selected molecules from laminin, fibronectin and collagen will be immobilized at the adhesive areas, after photo-ablation of the non-adhesive polymer. This step may be conventionally carried out by putting, for a duration of the order of one hour, the substrate into contact with a buffered aqueous solution of the desired ligand, at a concentration typically ranging from 0.1 to 100 μg/mL.

The substrates according to the invention may be used in various applications in cell biology which require immobilization of cell(s) on a substrate, such as cell cultivation, cytometry, drug screening, toxicology, diagnosis.

The object of the present invention is also a method for analyzing in vitro at least one cell comprising the following steps:

adhesion of a cell on an adhesive pattern of a substrate according to the invention,

cultivation of said cell,

analysis by optical imaging of said cell adhered onto the substrate.

Such a method advantageously comprises after the analysis step, detachment of the cell by varying an environmental parameter causing deployment of the non-adhesive polymer. The variation used preferably corresponds to a variation in temperature or light illumination as mentioned earlier and will be adapted to the polymers used.

The substrates according to the invention have the advantage of being reusable. The detachment process of the cells does not alter the substrate which has very high stability. Adhesion by adsorption of the cells on the adhesive areas is reversible, which is a definite advantage, as compared with prior techniques.

Analysis by optical imaging may use any automated technique, notably epifluorescence techniques such as those notably described in application EP 1 664 266.

The cells may be directly cultivated on the substrates according to the invention. Cell cultivation on substrates is subject to standard cell biology procedures: in particular the culture medium should be adapted to the investigated cell type. For example, it is possible to use a Dulbecco-modified Eagle medium (DMEM) containing 10% of bovine fetal serum, and 0.2% of penicillin and streptomycin. The cultivation may be achieved in a humidified sowing of the cells may be accomplished at a density ranging from 5,000 to 50,000 cells/cm². This density will preferably be adapted to the density of the patterns on the substrate for example, 10,000 cells per cm² for a distance between patterns of 100 μm). Most often, the adhesion and the cultivation of the cells are accomplished in an aqueous culture medium at 37° C., in the absence of light. The cell culture may be made in the presence of a drug or molecule, the activity of which is desirably tested.

The examples hereafter give the possibility of illustrating the invention but have no limiting nature.

The elaboration of brushes of poly(N-isopropylacrylamide) (PNIPAM) grafted on glass or silicon oxide substrates, and in which adhesive patterns are made by photo-ablation with deep UV is described in a detailed way. Next, the results obtained will be given, relating to:
cultivation and adhesion of cells on these surfaces,
an exemplary application of statistical analysis relating to spatial localization of a protein involved in cell adhesion,
detachment of the cells of the substrate, induced by a variation in temperature.

Characterization Methods and Instruments

Observation of cells were carried out on a Nikon Ti-E microscope equipped with a temperature-regulated incubation enclosure, giving the possibility of accomplishing imaging in transmission, by epifluorescence or in a bright field. The images of the morphology of the cells in phase contrast microscopy were obtained on an Olympus IX71 microscope with a 4× or 10× objective, immediately upon exiting the incubator, or else after binding the cells with paraformaldehyde. The temperature-induced cell detachment experiments were carried out on the Nikon microscope above, by setting the temperature of the weathering enclosure to 30° C. and by tracking the morphology of the cells over time.

The PNIPAM brushes elaborated on a silicon wafer were characterized by measuring their dry thickness, by means of a monochromatic ellipsometer (628 nm) with a rotating quarter wavelength slide.

Elaboration and Characterization of the Brushes

Brushes of poly(N-isopropylacrylamide) (PNIPAM) were grafted on glass substrates (specimen slides) and oxidized surfaces of substrates (wafers) of silicon, by the radical polymerization technique with atom transfer, according to a procedure inspired from the one described in the article of Malham and Bureau (Langmuir 26, 4762-4768 (2010)). The samples obtained on a silicon wafer were used for characterizing the thickness of the brushes by ellipsometry. The brushes deposited on glass slides were used for microscopy experiments on the achieved cell cultures.

Reagents: The N-isopropylacrylamide monomer was purified by recrystallization from hexane in order to suppress the polymerization inhibitor which stabilizes it during its marketing. The other reagents and solvent, in particular 3-aminopropyl-triethoxysilane (APTES), triethylamine (TEA), copper chloride (CuCl), 1,1,7,7-pentamethyldiethylenetriamine (PMDETA) and 2-bromo-2-methylpropionyl bromide (BMPB) were obtained in pure or ultra-pure quality (>98%) and used as such. All the aqueous solutions were prepared with ultra-pure water.

The glass and silicon substrates were first cleaned with successive rinses with acetone, ethanol and water, and then immersed for 15 minutes in an aqueous sodium hydroxide solution of concentration 1M, and finally rinsed with water.

The substrates were then immersed in an aqueous APTES solution at a concentration of 2.10⁻⁴ M for 1 minute. The APTES solution was prepared beforehand and stirred for one to two hours before immersion of the samples, in order to hydrolyze the ethoxysilane groups of APTES with view to grafting on the surfaces. This step gives the possibility of forming on the substrates a layer exposing NH₂ functions at the surface. The change in the concentration of APTES or in the immersion time affects the surface density of these NH₂ groups, which sets the final density of the polymeric brush. The conditions mentioned above were identified as allowing sufficient density to be obtained in order to ensure the sought anti-adhesive nature, regardless of temperature. Moreover, it was seen that with a brush elaborated on an APTES solution and a concentration of 2.10⁻⁵ M, the obtained density of the order of 0.05 chains/nm², does not give the possibility of ensuring the anti-adhesive function.

The substrates were then put into contact for one minute with a BMPB solution (250 μL) in dichloromethane (20 mL) in the presence of TEA (1.2 mL). This step allows functionalization of the NH₂ group by grafting BMBP molecules thereon, which expose at the surface the C—Br functions used as an initiator for the NIPAM polymerization reaction.

A solution of NIPAM (1 g) and PMDETA (150 μL) in water (20 mL) was prepared in a flask closed by a septum plug and purged with dissolved oxygen by argon bubbling for 30 minutes. 25 mg of CuCl were then added, and the solution was then stirred for a few minutes until a homogeneous solution of a green color was obtained. The substrates were then put into contact, at room temperature, with the solution for a time varying from 30 seconds to 10 minutes, and then abundantly rinsed with water before being dried in a nitrogen gas flow. This immersion time corresponds to the polymerization duration, which sets the number of monomers per chain and therefore their length.

The brushes elaborated under these conditions on an oxidized silicon wafer, were characterized by measuring by ellipsometry, their dry thickness, i.e. once cleared of the solvent used during the grafting. FIG. 2 shows the increase in this dry thickness with the polymerization time, which expresses the increase in the length of the grafted macromolecules. All the brushes elaborated within the thickness range appearing in FIG. 2 (between 15 and 65 nm in thickness) have demonstrated excellent anti-adhesive properties.

Making the Adhesive Patterns

The adhesive patterns on the brushes of PNIPAM were made by photo-ablation with deep UV. The illumination device used consists in four low pressure mercury lamps (Heraeus NobleLight NIQ 60/35XL, 60W, radiation with a wavelength <200 nm) placed side by side horizontally in a stainless steel enclosure. The samples were all insolated at a vertical distance of 9 cm from the lamps.

Under these conditions, the photo-ablation kinetics were first of all characterized by tracking by ellipsometry the time-dependent change in the dry thickness of a brush grafted on a silicon wafer, according to the insolation time. FIG. 3 shows an example of this evolution for a brush with an initial thickness of 65 nm. It is seen that after five minutes of illumination, the brush has been completely withdrawn from the substrate.

The dry PNIPAM brushes were put into direct contact with a photomask bearing transparent patterns with various geometries (annular, triangular, rectangular or V-shaped). These quartz/chromium masks were obtained from Toppan Photomasks Inc. Texas, USA. The insolation time was selected between 5 and 10 minutes, without this selection significantly affecting the quality of the patterns.

Adsorption of Proteins

In order to reveal the shape and the definition of the patterns made, adsorption experiments with a fluorescent protein were conducted. A solution containing 20 μg/mL of fibronectin/fibrinogen-Alexa fluor 546 nm (Invitrogen) in a 10 mM d'Hepes buffer solution (pH 8.5) was prepared. The substrates prepared previously bearing microstructured PMIPAM brushes were put into contact with the solution and incubated for one hour, away from light, and then rinsed with a saline buffer solution of PBS (Phosphate Buffered Saline). FIGS. 4 and 5 give examples of patterns made, viewed by microscopy with epifluorescence, by detecting the signal from the adsorbed proteins. These figures demonstrate:

(i) the good anti-adhesive properties of the PNIPAM brushes (no fluorescence detected outside the adhesive patterns).

(ii) the homogeneity of the large scale patterns (of the order of a mm²) obtained with the described method.

(iii) the good resolution and the variety in the shape of the patterns.

Cultivation of Cells

Cells (fibroblasts) of the REF52 and MEF type were cultivated on substrates elaborated in a humidified atmosphere containing 5% of carbon dioxide gas and 95% of air, in a Dulbecco-modified Eagle medium (DMEM) containing 10% of bovine fetal serum and 0.2% of penicillin and streptomycin. The REF52s were cultivated on all the elaborated substrates. The MEFs were only cultivated on substrates coated with a PNIPAM brush with a dry thickness of 65 nm and with a density of 0.5 chain/nm². The cells were sown on surfaces with a density of 50,000 cells/cm². After 30 minutes, the non-adhering cells located between the patterns were removed by rinsing with pure culture medium.

The cells were sown either on the microstructured surfaces which had not been subject to any prior treatment, or on substrates on which fibronectin/fibrinogen-alexa has been deposited beforehand. Good spreading of the cells on the patterns was observed in both cases (see FIGS. 6 and 9), with differences on the time required for obtaining this spreading: two hours of cultivation were sufficient on the substrates pre-coated with fibronectin, as well as about ten hours required in the case of sowing on substrates exposing the naked glass surface in the adhesive areas.

After spreading of the cells, the latter were fixed with paraformaldehyde.

Results Obtained

1—Statistical Analysis of the Cell Responses

FIG. 6 shows an example, obtained by transmission optical microscopy of a network formed by REF52 cells adhered on a V-shaped pattern. The REF52s are cells expressing a fluorescent form of paxillin (paxillin-YFP), a protein associated with the cell/substrate adhesion mechanism. It was shown in prior work that during the cell adhesion on a pattern having apices, different intracellular proteins involved in the interactions with the substrate aggregate at these apices (Theryet al, Proc. Natl. Acad. Sci. USA. 103 (2006) pp 19771-19776). We were able to show this aggregation phenomenon with paxillin by constructing the average fluorescence image obtained by superposing the images of 11 different cells adhered on a V-shaped pattern (FIG. 8). The benefit of the construction of this average image is to increase the signal/noise ratio in order to show the spatial localization of the protein, a localization which does not appear or very little on the fluorescence image of a single cell (FIG. 7). This example of determination of the intracellular organisation shows that the microstructured substrate made here actually allows application of a statistical analysis based on the reproducibility of the shape of the immobilized cells on the surface.

2—Obtaining Different Cell Forms

The photo-ablation method described earlier was used for making different shapes of adhesive patterns on the PNIPAM surfaces. FIG. 9 shows three examples of adhesive patterns (triangular, annular and rectangular patterns) onto which the cells will adhere following the geometry imposed by the substrate.

3—Detachment of the Cells Induced by a Variation in Temperature

The detachment of the cells is induced when the temperature is lowered below the TCIS of the PNIPAM, which has the value of 32° C. The physical mechanism which causes the detachment of the cells is the change in conformation of the PNIPAM chains grafted on the surface upon lowering of the temperature. This mechanism is schematized in FIG. 1.

FIGS. 10 and 11 show, for two forms of initial cells and for both cell types tested, a time sequence of images obtained after reducing the temperature of the atmosphere to T=30° C. Theses sequences show the retraction and the rounding of the cells which indicates their detachment from the substrate. The detachment occurs over a period ranging from 25 to 45 minutes.

4—Stability of the Patterns—Possibility of Reusing the Substrates

The detachment of the cells induced by temperature gives the possibility of not only recovering in solution the population of the study itself, but also of reusing the microstructured substrate for performing other cultivations. FIG. 12 shows images corresponding to three successive cultivations, between which the cells have been detached by lowering the temperature down to room temperature and rinsing with the pure culture medium. It is seen that the occupation level of the network of patterns (number of cells per unit surface) is slightly affected between the first cultivation and the two following ones (this level passes from 100 cells/mm² to about 80 cells/mm² between the first cultivation and the two following ones). Moreover, FIG. 12(d) shows that no cell remains adhered on the substrate between two cultivations.

The possibility of reusing the substrates for several cultivations also demonstrates the good stability of the adhesive patterns made with the method described in this example.

1- A substrate for adhesion of at least one animal or human cell on the surface of which is generated an adhesion pattern for at least one animal or human cell, said pattern including at least two adhesive areas to said cell which are spaced from each other by a non-adhesive area to said cell on which a non-adhesive polymer to said cell is immobilized, characterized in that the adhesive areas of an adhesive pattern are placed so that the cell may adhere, under adhesion conditions, onto the adhesive areas by forming a bridge over the non-adhesive polymer, the polymer has a conformation which may be modified by changing a parameter of the environment in which the substrate is found and appears as a so-called compacted conformation, under adhesion conditions of the cell, in order to be deployed in a so-called deployed conformation by modification of a parameter of the environment in which the substrate is found.

2- The substrate according to claim 1, characterized in that the polymer defines a layer for which the thickness may be increased relatively to a reference thickness when the substrate is placed in pure water at 37° C. and in the absence of light, by modification of a parameter of the environment in which the substrate is found.

3- The substrate according to claim 1, characterized in that the ratio between the non-adhesive area and the total surface area of the pattern is between 50 and 90%.

4- The substrate according to claim 1, characterized in that the non-adhesive polymer forms a layer for which the average thickness may be increased by at least a factor of 2, relatively to its average reference thickness when the substrate is placed in pure water at 37° C. and in the absence of light, or relatively to its average thickness in its compacted conformation, by means of the modification of a parameter of the environment in which the substrate is found.

5- The substrate according to claim 1, characterized in that the polymer has a conformation which may be modified by varying the temperature and in particular by lowering the temperature.

6- The substrate according to claim 5, characterized in that the non-adhesive polymer is a heat-sensitive polymer having in water a lower critical solubility temperature which belongs to the range from 25 to 35° C.

7- The substrate according to claim 5, characterized in that the polymer is selected from poly(N-isopropylacrylamide), poly(oligo(ethylene glycol)methacrylate) and poly(N,N-dimethyl-aminoethyl methacrylate).

8- The substrate according to claim 1, characterized in that the polymer has a conformation which may be modified by varying the light illumination.

9- The substrate according to claim 8, characterized in that the polymer is poly{N,N-dimethylaminoethylmethacrylate-co-4-methyl-[7-(methacryloyl)oxyethyloxy]coumarin}.

10- The substrate according to claim 1, characterized in that the non-adhesive polymer is immobilized on the surface of the substrate through a covalent bond.

11- The substrate according to claim 1, characterized in that the non-adhesive polymer is immobilized with a density of polymeric chains belonging to the range from 0.1 to 1 chain per nm².

12- The substrate according to claim 1, characterized in that the non-adhesive polymer is immobilized according to a brush of polymeric chains, each of the polymeric chains being bound to the surface of the substrate through one of its ends.

13- The substrate according to claim 1, characterized in that the non-adhesive polymer appears in the form of polymeric chains comprising from 50 to 10,000 monomer units.

14- The substrate according to claim 1, characterized in that the size of the pattern is adapted so as to allow adhesion of a single individual cell.

15- The substrate according to claim 1, characterized in that the maximum distance generated by the non-adhesive area separating two adhesive areas of a pattern, is less than 50 μm, preferably belongs to the range from 5 to 20 μm.

16- The substrate according to claim 1, characterized in that molecules selected from laminin, fibronectin and collagen are immobilized on the adhesive areas of the adhesive pattern.

17- The substrate according to claim 1, characterized in that the adhesive areas of the adhesive patterns are connected together in order to form an open or closed form or are isolated from each other.

18- The substrate according to claim 1, characterized in that the adhesive areas of the adhesive patterns form a broken line or curve which may be closed for example delimiting a polygon or a ring or open, for example with the shape of a V, U, C or L, or else correspond to two spaced apart lines or to a line and one or several spaced apart dots.

19- The substrate according to claim 1, characterized in that a network of identical adhesive patterns as defined in the preceding claims is present at its surface.

20- The substrate according to claim 19, characterized in that the non-adhesive polymer immobilized on the non-adhesive areas of the adhesive patterns is also immobilized on the regions separating two consecutive adhesive patterns.

21- The substrate according to claim 1, characterized in that the substrate consists of glass, silanized glass, silica or silicon oxidized at the surface.

22- A method for preparing a substrate according to claim 1, wherein the selected non-adhesive polymer is immobilized notably through a covalent bond, on the total surface of the substrate and photo-ablation of the non-adhesive polymer is then achieved by photolithography at the areas corresponding to the adhesive areas of the adhesive patterns.

23- The preparation method according to claim 22, characterized in that the immobilization of the selected non-adhesive polymer at the surface of the substrate is achieved by controlled radical polymerization of at least one monomer on an immobilized polymerization initiator, preferably through a covalent bond, at the surface of the substrate.

24- The preparation method according to claim 22, characterized in that molecules selected from laminin, fibronectin and collagen are immobilized at the adhesive areas after photo-ablation of the non-adhesive polymer.

25- A method for detachment in vitro of at least one cell having adhered beforehand on the adhesive pattern(s) of a substrate according to claim 1, characterized in that the detachment of the cell is achieved by varying an environmental parameter causing deployment of the non-adhesive polymer.

26- A method for in vitro analysis of at least one cell comprising the following steps:
adhesion of a cell on an adhesive pattern of a substrate according to claim 1,
cultivation of said cell,
analysis by optical imaging of said cell adhered onto the substrate.

27- The analysis method according to claim 26, characterized in that it comprises, after the analysis step, detachment of the cell by varying an environmental parameter causing deployment of the non-adhesive polymer.
